(19) United States
(12) Patent Application Publication
(10) Pub. No.: US 2026/0283337 A1
(43) Pub. Date: Sep. 24, 2026

(54) WEARABLE CLIP FOR FLUID CONTAINERS

(71) Applicant: Bob Richards, St. George, UT (US)

(72) Inventor: Bob Richards, St. George, UT (US)

(21) Appl. No.: 19/571,671

(22) Filed: Mar. 19, 2026

Related U.S. Application Data
(60) Provisional application No. 63/774,281, filed on Mar. 19, 2025.

(51) Int. Cl. A45F 5/02; A61J 1/16

(52) U.S. Cl. CPC A45F 5/02; A61J 1/16

(57) ABSTRACT

A wearable clip for fluid containers comprises a jaw comprising a first member and a second member, a plurality of teeth disposed in the jaw, wherein a first row of teeth is disposed on the first member and a second row of teeth is disposed on the second member, the first row of teeth and the second row of teeth configured to mate with one another when the jaw is in a closed position, a hinge interposed between the first member and the second member, a lever configured to actuate the jaw about the hinge, a spring configured to bias the jaw in the closed position, and a bag support coupled to the second member and configured to receive a fluid container.

### CROSS-REFERENCE TO RELATED APPLICATIONS

This application claims the benefit of U.S. Provisional Application Ser. No. 63/774,281, filed on Mar. 19, 2025, which is incorporated herein by reference in its entirety.

### FIELD OF INVENTION

The present disclosure relates to fluid containers, such as intravenous bags and accessories for medical applications.

### BACKGROUND

Healthcare providers and first responders frequently administer intravenous (IV) solutions to patients in a variety of settings. In medical facilities, IV bags are typically suspended from dedicated bag stands or poles that maintain the fluid container at an elevated position relative to the patient, allowing gravity to facilitate fluid flow through the IV tubing to the patient.

However, many patient care scenarios occur outside of traditional medical facilities where dedicated IV stands or poles are not readily available. Such scenarios include emergency response situations, patient transport, field medicine, and rescue operations in challenging terrain such as mountains or canyons. In these circumstances, a healthcare provider typically holds the IV bag with one hand elevated above the patient to maintain proper fluid flow.

This manual approach to IV bag support presents several challenges. When a provider uses one hand to hold an IV bag, their ability to render additional care to the patient is diminished by only having one hand available. Additionally, the provider may experience physical strain from holding the IV bag in an elevated position for extended periods. Further, the risk of injury to the provider may increase when attempting to maneuver or transport a patient while simultaneously holding an IV bag, particularly in rough or uneven terrain.

The logistics of patient care may also be affected when manual IV bag support is employed. In some situations, an additional responder may be assigned solely to hold the IV bag, which increases the personnel requirements and associated costs for patient care. This approach may not be practical in many emergency scenarios where personnel resources are limited.

Various devices exist for supporting medical fluid containers in different contexts. Some devices are designed for attachment to stationary structures such as bed rails, poles, or wall-mounted fixtures. Other devices are configured for attachment to the fluid containers themselves. However, the range of available solutions for hands-free IV bag support in mobile patient care scenarios remains limited.

Accordingly, there is a need for an apparatus that can maintain a fluid container at an elevated position relative to a patient while allowing a healthcare provider to move freely and use both hands for patient care. The present disclosure seeks to solve these and other problems.

### SUMMARY

This summary is provided to introduce a selection of concepts in a simplified form that are further described below in the detailed description. This summary is not intended to identify key features or essential features of the claimed subject matter.

According to some aspects of the present disclosure, a wearable clip for fluid containers is provided. The wearable clip for fluid containers includes a jaw, a plurality of teeth disposed in the jaw, a lever and hinge configured to actuate the jaw, a spring configured to bias the hinge in a first direction to maintain the jaw in a closed position, and a bag support. The bag support is configured to receive or support a fluid container.

According to some aspects of the present disclosure, the wearable clip for fluid containers may include one or more of the following features. The bag support may comprise a carabiner. The jaw may comprise a first member having a first row of teeth and a second member having a second row of teeth, wherein the first row of teeth and the second row of teeth are configured to mate with one another when the jaw is in the closed position. The first member may comprise a first arm that extends to form the lever. The second member may comprise a second arm, with the hinge interposed between the first arm and the second arm, the hinge functioning as a fulcrum for the lever. The carabiner may be coupled to the second arm. The wearable clip for fluid containers may be configured to clamp onto an article of clothing worn by a healthcare provider. The fluid container may comprise an intravenous solution bag.

According to some aspects of the present disclosure, a method of using a wearable clip for fluid containers is provided. The method includes actuating a lever to open a jaw of the wearable clip, the jaw comprising a plurality of teeth. The method further includes placing an article of clothing between the teeth of the jaw. The method further includes releasing the lever, wherein a spring forces the jaw closed, thereby clamping the article of clothing within the jaw. The method further includes coupling a fluid container to a bag support of the wearable clip.

According to some aspects of the present disclosure, the method may include one or more of the following features. The bag support may comprise a carabiner. The method may further include placing tubing through the carabiner during patient transport. The fluid container may comprise an intravenous solution bag. The article of clothing may be worn by a healthcare provider, and the wearable clip may be secured at an elevated position relative to a patient such that gravity facilitates fluid flow from the fluid container to the patient.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a front, side perspective view of a wearable clip for fluid containers, according to aspects of the present disclosure.

FIG. 2 illustrates a bottom, side perspective view of the wearable clip for fluid containers, according to aspects of the present disclosure.

FIG. 3 illustrates a side elevation view of the wearable clip for fluid containers, according to aspects of the present disclosure.

FIG. 4 illustrates a front perspective view of the wearable clip for fluid containers in use, according to aspects of the present disclosure.

FIG. 5 illustrates a front, side perspective view of the wearable clip for fluid containers in use, according to aspects of the present disclosure.

FIG. 6 illustrates a front perspective view of the wearable clip for fluid containers in use with an IV bag, according to aspects of the present disclosure.

### DETAILED DESCRIPTION

The following descriptions depict only example embodiments and are not to be considered limiting in scope. Any reference herein to “the invention” is not intended to restrict or limit the invention to exact features or steps of any one or more of the exemplary embodiments disclosed in the present specification. References to “one embodiment,” “an embodiment,” “various embodiments,” and the like, may indicate that the embodiment(s) so described may include a particular feature, structure, or characteristic, but not every embodiment necessarily includes the particular feature, structure, or characteristic. Further, repeated use of the phrase “in one embodiment,” or “in an embodiment,” do not necessarily refer to the same embodiment, although they may.

Reference to the drawings is done throughout the disclosure using various numbers. The numbers used are for the convenience of the drafter only and the absence of numbers in an apparent sequence should not be considered limiting and does not imply that additional parts of that particular embodiment exist. Numbering patterns from one embodiment to the other need not imply that each embodiment has similar parts, although it may.

Accordingly, the particular arrangements disclosed are meant to be illustrative only and not limiting as to the scope of the invention, which is to be given the full breadth of the appended claims and any and all equivalents thereof. Although specific terms are employed herein, they are used in a generic and descriptive sense only and not for purposes of limitation. Unless otherwise expressly defined herein, such terms are intended to be given their broad, ordinary, and customary meaning not inconsistent with that applicable in the relevant industry and without restriction to any specific embodiment hereinafter described. As used herein, the article “a” is intended to include one or more items. When used herein to join a list of items, the term “or” denotes at least one of the items, but does not exclude a plurality of items of the list. For exemplary methods or processes, the sequence and/or arrangement of steps described herein are illustrative and not restrictive.

It should be understood that the steps of any such processes or methods are not limited to being carried out in any particular sequence, arrangement, or with any particular graphics or interface. Indeed, the steps of the disclosed processes or methods generally may be carried out in various sequences and arrangements while still falling within the scope of the present invention.

The term “coupled” may mean that two or more elements are in direct physical contact. However, “coupled” may also mean that two or more elements are not in direct contact with each other, but yet still cooperate or interact with each other.

The terms “comprising,” “including,” “having,” and the like, as used with respect to embodiments, are synonymous, and are generally intended as “open” terms (e.g., the term “including” should be interpreted as “including, but not limited to,” the term “having” should be interpreted as “having at least,” the term “includes” should be interpreted as “includes, but is not limited to,” etc.).

The present disclosure relates to a wearable clip for fluid containers that may be secured to an article of clothing worn by a healthcare provider or first responder. The wearable clip may allow the provider to maintain a fluid container, such as an intravenous (IV) solution bag, at an elevated position relative to a patient without requiring the provider to manually hold the fluid container. In some cases, the wearable clip may enable gravity-assisted fluid delivery from the fluid container to the patient while the provider retains the use of both hands to render aid, transport the patient, or perform other tasks.

In various scenarios, healthcare providers and first responders may need to administer IV solutions to patients in locations where traditional IV bag stands or poles are not readily available. Such scenarios may include emergency response situations, field medical care, mountain or canyon rescues, patient transport, or other circumstances where mobility and hands-free operation are beneficial. The wearable clip for fluid containers described herein may address these scenarios by providing a means for securing a fluid container to the provider's clothing at a position elevated above the patient.

The wearable clip may reduce the number of personnel required to provide care to a patient by eliminating the need for a dedicated person to hold the fluid container. The wearable clip may also reduce the risk of injury to providers who might otherwise attempt to hold a fluid container while simultaneously maneuvering or transporting a patient, particularly in challenging terrain or confined spaces. In some cases, the wearable clip may accommodate various types of fluid containers beyond IV bags, including hydration containers or other bags equipped with fluid tubes or straws.

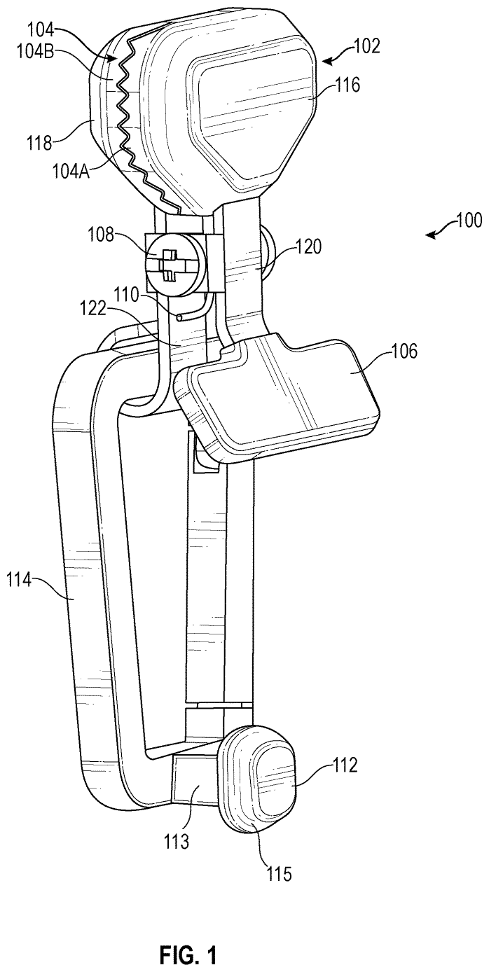

Referring to FIG. 1, a wearable clip for fluid containers 100 comprises a jaw 102, a plurality of teeth 104 in the jaw 102, a lever 106 and hinge 108 configured to actuate the jaw 102, a spring 110 configured to bias the jaw 102 toward a closed position via the hinge, and a bag support 112. In some embodiments, the bag support 112 may further comprise, or be coupled to, a carabiner 114.

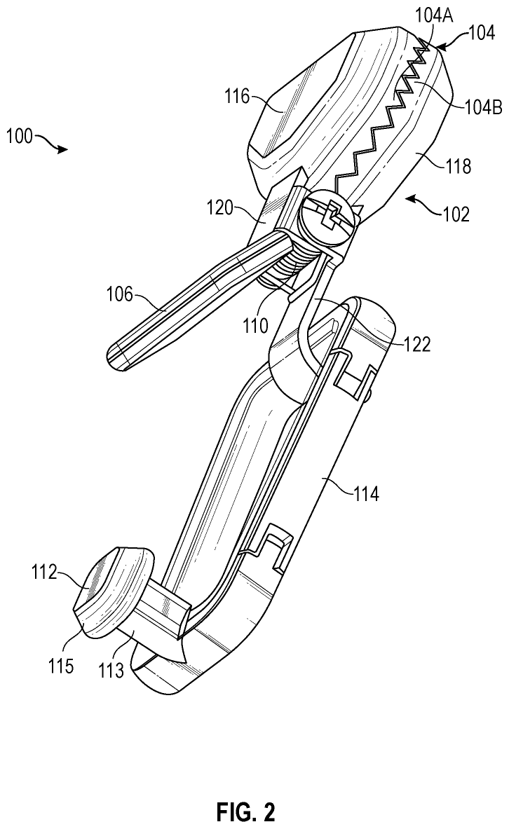

As understood, the jaw 102 may comprise a first member 116, the first member 116 comprising a first row of teeth 104A, and a second member 118 comprising a second row of teeth 104B. The first row of teeth 104A and second row of teeth 104B are configured to mate with one other, effectively closing the jaw 102. The first member 116 may comprise a first arm 120 that extends to form the lever 106. The second member 118 may comprise a second arm 122, with the hinge 108 interposed between the first and second arms 120, 122, with the hinge 108 functioning as the fulcrum for the lever 106. As shown, the hinge 108 may simply be a threaded screw, bolt, or pin that may also function to couple the first arm 120 to the second arm 122, with the spring 110 positioned over the hinge 108, as best shown in FIG. 2.

The spring 110 may be configured to force the lever 106 and the first arm 120 in a first direction, thereby maintaining the first member 116 and the second member 118 in contact with one another with the first row of teeth 104A and the second row of teeth 104B mated.

As further shown in FIG. 1, a bag support 112 may be coupled to the second arm 122. The bag support 112 may extend downward from the second arm 122. The bag support 112 may comprise or otherwise incorporate a carabiner 114. The carabiner 114 may provide a means for attaching fluid containers, such as IV bags, or associated tubing, to the wearable clip for fluid containers 100.

Referring to FIG. 2, a bottom, side perspective view of the wearable clip for fluid containers 100 further illustrates the spatial arrangement of the components. From this perspective, the jaw 102 is visible at the upper portion of the wearable clip for fluid containers 100, with the first member 116 and the second member 118 in a closed, default position. The teeth 104, comprising the first row of teeth 104A on the first member 116 and the second row of teeth 104B on the second member 118, are configured to interlock when the jaw 102 is in a closed position.

With continued reference to FIG. 2, the spring 110 is positioned between the first arm 120 and the second arm 122. The spring 110 may exert a biasing force that urges the first member 116 and the second member 118 together, thereby maintaining the jaw 102 in a closed position with the first row of teeth 104A mated with the second row of teeth 104B. In some cases, the spring 110 may be a torsion spring or other spring type configured to provide a closing force on the jaw 102.

As further shown in FIG. 2, the lever 106 may be configured to actuate the jaw 102. When a user presses the lever 106, actuating it toward the second arm 122 and carabiner 114, the lever 106 may compress the spring 110, thereby separating the first row of teeth 104A from the second row of teeth 104B and opening the jaw 102. Upon release of the lever 106, the spring 110 may return to an uncompressed or less compressed state, forcing the jaw 102 closed. This arrangement may allow a provider to open the jaw 102 with one hand by pressing the lever 106, position an article of clothing between the teeth 104, and release the lever 106 to clamp the article of clothing within the jaw 102. The bottom perspective view also shows the bag support 112 extending outwardly from the carabiner 114 coupled to the second arm 122. While a carabiner 114 is shown, it will be appreciated that it is not required, and the second arm 122 may extend beyond the length of the lever 106 and may comprise the bag support 112 thereon.

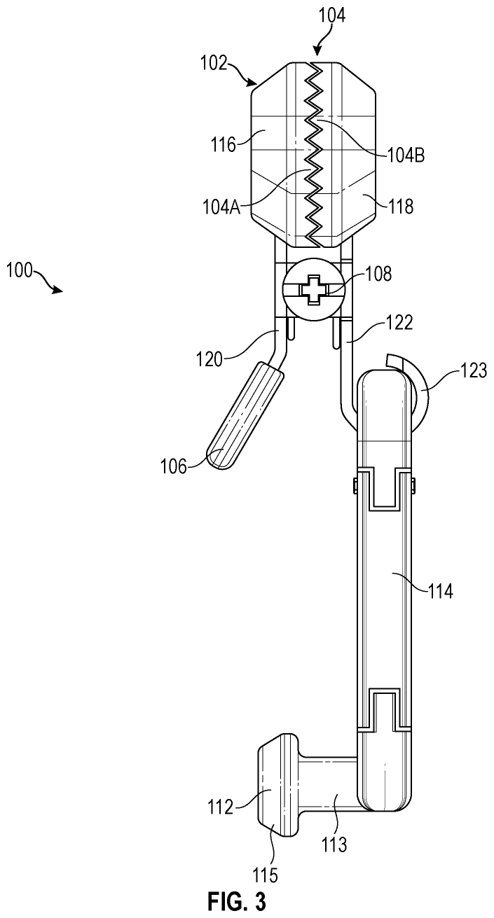

Referring to FIG. 3, a side elevation view of the wearable clip for fluid containers 100 illustrates the vertical arrangement of the components. The jaw 102 is positioned at the upper portion of the wearable clip for fluid containers 100. The jaw 102 comprises the first member 116 and the second member 118, with the teeth 104 arranged in the first row of teeth 104A on the first member 116 and the second row of teeth 104B on the second member 118. The first row of teeth 104A and the second row of teeth 104B may be configured to interlock when the jaw 102 is in a closed position.

With continued reference to FIG. 3, the first member 116 extends to form the first arm 120, which connects to the lever 106. The second member 118 extends to form the second arm 122. The hinge 108 is interposed between the first arm 120 and the second arm 122, and the hinge 108 may serve as a fulcrum for the lever 106 to actuate the jaw 102. The side elevation view demonstrates the relationship between the clamping mechanism at the top of the wearable clip for fluid containers 100 and the bag support structure at the bottom of the wearable clip for fluid containers 100.

As further shown in FIG. 3, the second arm 122 extends downward from the hinge 108 and connects to the bag support 112. The bag support 112 is coupled to the carabiner 114, which is positioned at a lower portion of the wearable clip for fluid containers 100. The carabiner 114 may be configured to receive and support a fluid container, such as an IV bag. The side elevation view illustrates how the wearable clip for fluid containers 100 may provide a vertical arrangement wherein the jaw 102 at the upper portion clamps onto an article of clothing while the bag support 112 and the carabiner 114 extend downward to support a fluid container at a position below the clamping point. This vertical arrangement may allow a fluid container attached to the carabiner 114 to hang freely below the point of attachment to the provider's clothing.

As shown, the second arm 122 may comprise a hook 123 configured to allow the carabiner 114 to pivot therein, allowing for the carabiner 114 to adjust based on gravity and body positioning. The bag support 112 may comprise a neck 113 and a head 115. The head 115 may aid in preventing a bag attached thereon from inadvertently falling off.

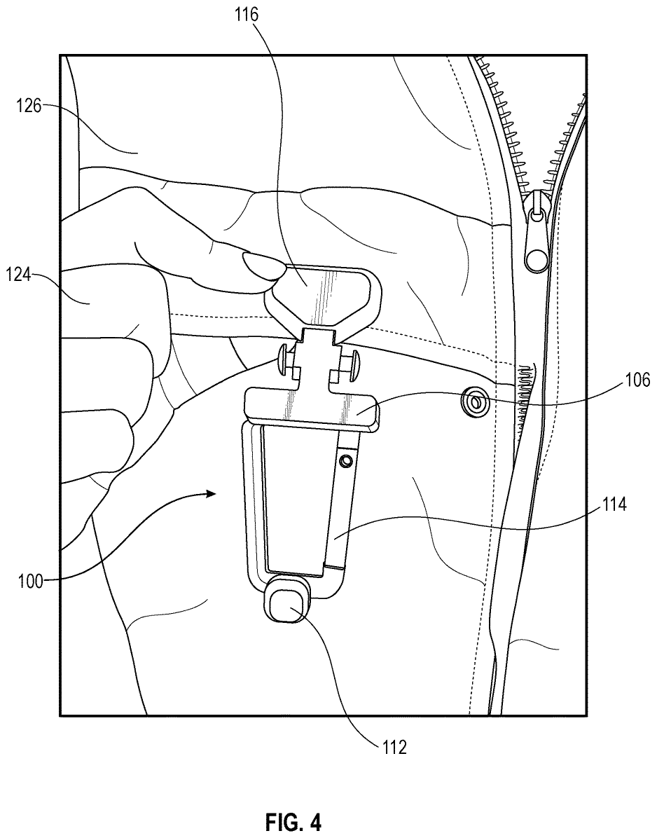

Referring to FIG. 4, the wearable clip for fluid containers 100 is shown attached to clothing 126 worn by a provider 124. The provider 124 may be a healthcare provider, first responder, or other individual administering care to a patient. The clothing 126 may be a jacket, shirt, vest, or other garment worn by the provider 124.

The provider 124 may secure the wearable clip for fluid containers 100 to the clothing 126 by forcing the lever 106 toward the second arm 122, causing the first arm 120 to pivot on the hinge 108, thereby opening the jaw 102. In an open position, the first member 116 is separated from the second member 118. With the jaw 102 in an open position, the first row of teeth 104A and the second row of teeth 104B may be spaced apart to receive the clothing 126 therebetween.

The provider 124 may position the clothing 126 such that a portion of the fabric of the clothing 126 is disposed between the first row of teeth 104A and the second row of teeth 104B. In some cases, the provider 124 may attach the wearable clip for fluid containers 100 to an upper portion of the clothing 126, such as near a shoulder, collar, or chest area of the provider 124, to position the wearable clip for fluid containers 100 at an elevated location relative to a patient.

Upon releasing the lever 106, the spring 110 may force the jaw 102 closed. In other words, the spring 110 may urge the first member 116 toward the second member 118, causing the first row of teeth 104A to mate with the second row of teeth 104B. The teeth 104 may clamp the clothing 126 within the jaw 102, thereby securing the wearable clip for fluid containers 100 to the provider 124. The biasing force of the spring 110 may maintain the jaw 102 in the closed position, holding the clothing 126 between the teeth 104 with sufficient force to support a fluid container coupled to the bag support 112 or the carabiner 114. This configuration may allow the provider 124 to support a fluid container at an elevated position while retaining the use of both hands to render aid to a patient.

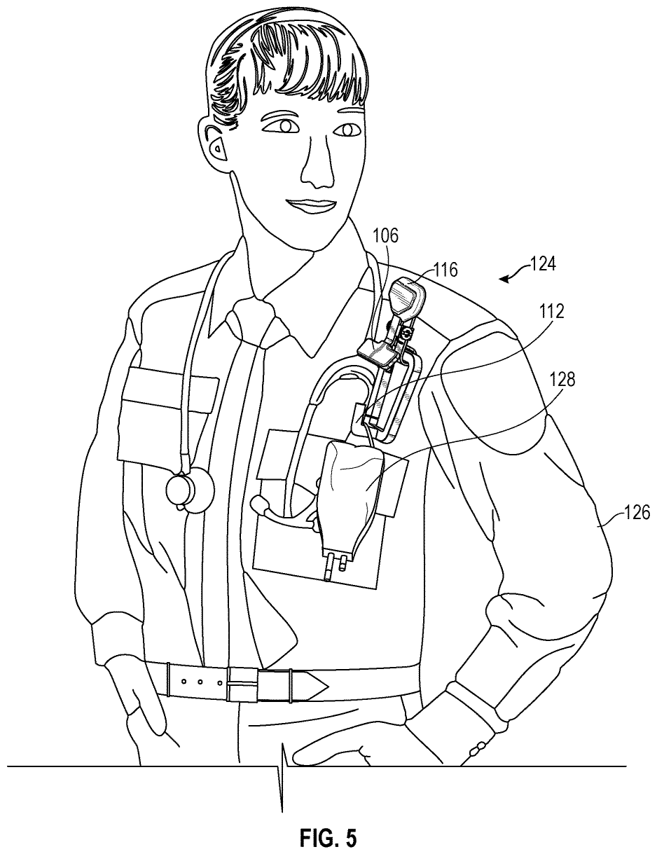

Referring to FIG. 5, the provider 124 is shown wearing the wearable clip for fluid containers secured to the clothing 126 at a shoulder area of the provider 124. The provider 124 is illustrated in a standing position with arms positioned at the sides of the provider 124, demonstrating how the wearable clip for fluid containers 100 may allow hands-free operation while the wearable clip for fluid containers 100 remains securely fastened to the clothing 126.

With continued reference to FIG. 5, the positioning of the wearable clip for fluid containers 100 at the shoulder area of the provider 124 may place the wearable clip for fluid containers 100 at an elevated location relative to a patient receiving care. In some cases, the patient may be positioned below the provider 124, such as when the patient is lying on a stretcher, gurney, or the ground. By securing the wearable clip for fluid containers 100 to an upper portion of the clothing 126, such as at the shoulder area, the provider 124 may maintain a fluid container at a higher elevation than the patient.

The elevated positioning of the wearable clip for fluid containers 100 on the clothing 126 of the provider 124 may enable gravity to facilitate fluid flow from a fluid container to the patient. When a fluid container, such as an IV bag 128, is coupled to the wearable clip for fluid containers 100, the fluid container may hang below the point of attachment at the shoulder area of the provider 124. The elevation difference between the fluid container and the patient may create a gravitational pressure differential that drives fluid from the fluid container through tubing to the patient. In some cases, the provider 124 may position the wearable clip for fluid containers 100 at various locations on the clothing 126 to adjust the elevation of the fluid container relative to the patient based on the specific care scenario or patient positioning.

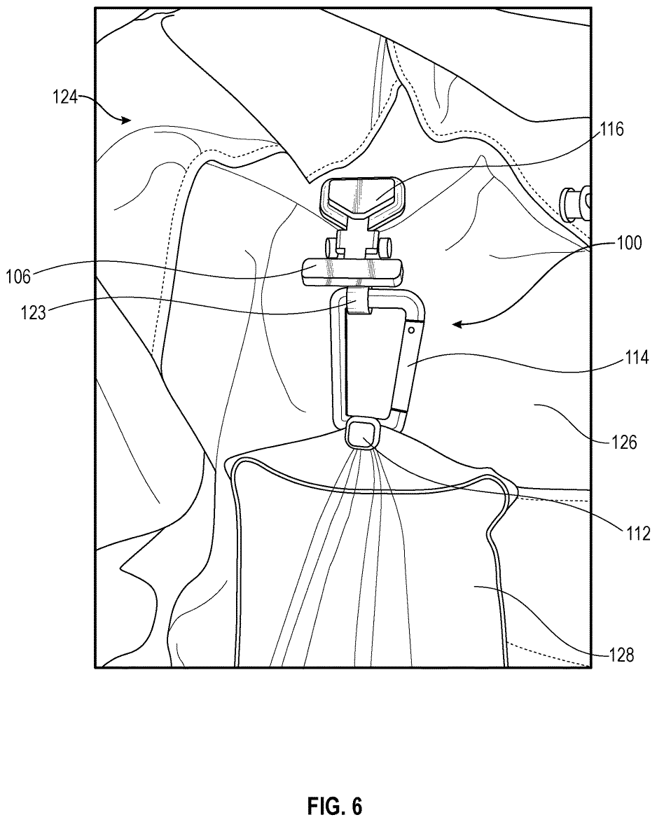

Referring to FIG. 6, the wearable clip for fluid containers 100 is shown attached to the clothing 126 worn by the provider 124, with an IV bag 128 coupled to the wearable clip for fluid containers 100. The IV bag 128 may be coupled to the carabiner 114 or the bag support 112 of the wearable clip for fluid containers 100. When attached to the bag support 112, as shown, the head 115 prevents unwanted withdrawal of the IV bag 128 from the bag support 112, the IV bag 128 being supported by the neck 113 of the bag support 112. The IV bag 128 hangs below the wearable clip for fluid containers 100, with tubing extending downward from a lower portion of the IV bag 128.

With continued reference to FIG. 6, the wearable clip for fluid containers 100 is secured to the clothing 126 at an elevated position on an upper body of the provider 124, such as near a shoulder or collar area. The positioning of the wearable clip for fluid containers 100 on the clothing 126 allows the IV bag 128 to hang at an elevated position relative to a patient positioned below the provider 124. The elevation of the IV bag 128 may enable gravity to facilitate fluid flow from the IV bag 128 through the tubing to the patient.

As a result, the provider 124 may maintain both hands free while the IV bag 128 remains coupled to the wearable clip for fluid containers 100. This configuration may allow the provider 124 to render aid to the patient, assist with patient transport, or perform other tasks without manually holding the IV bag 128. In some cases, the provider 124 may use both hands to maneuver or transport the patient while the wearable clip for fluid containers 100 supports the IV bag 128 at the elevated position.

In some cases, the carabiner 114 may be used to place tubing, cables, or other items through the carabiner 114 during patient transport. The carabiner 114 may provide additional support for tubing extending from the IV bag 128 or for other cables and items associated with patient care. This arrangement may be beneficial during mountain or canyon rescues, or in other scenarios where additional tubing support is advantageous. The carabiner 114 may accommodate multiple items passed through an opening of the carabiner 114, allowing the provider 124 to organize and secure various lines or cables while transporting the patient. This helps to ensure that the tubing and cables are not tangled, caught on objects or surfaces, and remain securely connected to their respective ports.

In some cases, the jaw 102 may be configured without teeth. The jaw may comprise any clamping means configured to grip an article of clothing with sufficient force to support a fluid container filled with fluids. For example, the jaw may include smooth clamping surfaces, textured surfaces, rubberized gripping surfaces, or other configurations that provide frictional engagement with fabric without discrete teeth. The clamping means may be configured to hold a standard IV bag filled with fluids, which may weigh approximately two and a quarter pounds (one kilogram) or more when full. In some cases, the clamping means may include pads, ridges, or other surface features that enhance grip on the clothing without requiring interlocking teeth.

In some cases, the bag support 112 may comprise the second arm 122 extending beyond the lever 106 and shaped or configured to support an IV bag 128 thereon without requiring a separate carabiner 114. The second arm 122 may extend downward past the hinge 108 and may terminate in a curved, hooked, or angled portion configured to receive a handle, loop, or hanging feature of an IV bag 128. In some cases, the second arm 122 may be bent or formed into a shape that allows an IV bag 128 to be hung directly on the second arm 122 without an intermediate attachment device. This configuration may simplify the wearable clip by reducing the number of components while still providing support for a fluid container.

In some cases, a ring, hook, or other member may be coupled to the second arm 122 for supporting an IV bag 128 thereon as an alternative to a carabiner 114. The ring may be a closed loop of material, such as metal or plastic, through which a handle or loop of an IV bag 128 may be passed. The hook may be an open or partially open curved member configured to receive and retain a portion of an IV bag 128. In some cases, the other member may comprise a clip, clasp, snap, or other fastening device configured to engage with an IV bag 128 or a hanging feature of an IV bag 128. These alternative configurations may provide different attachment options depending on the type of fluid container being used or the preferences of the provider.

In some cases, the wearable clip for fluid containers 100 may be used with fluid containers other than IV bags 128. The wearable clip for fluid containers 100 may support hydration containers, such as water bags or hydration bladders used for personal hydration. In some cases, the wearable clip for fluid containers 100 may support bags with a fluid tube or straw attached, allowing a user to drink from the container while the container remains secured to the wearable clip. The wearable clip may accommodate various sizes and shapes of fluid containers, provided the fluid container includes a handle, loop, or other feature that can be coupled to the bag support, carabiner, ring, hook, or other member of the wearable clip.

The components of the wearable clip for fluid containers 100 may work together to allow a provider to maintain a fluid container at an elevated position relative to a patient while the provider uses both hands for patient care. The jaw 102 and associated clamping mechanism (collectively 102, 104, 106, 108) may secure the wearable clip to the provider's clothing at an upper portion of the provider's body. The bag support 112, whether comprising a carabiner 114, an extended second arm, a ring, a hook, or another member, may support the fluid container below the point of attachment to the clothing. While the bag support 112 is shown and discussed as being coupled to or integrated with the second member 118, it will be appreciated that it may alternatively be coupled to the first member 116 or otherwise operatively coupled to the jaw 102 or clamping mechanism. The spring 110 may maintain the jaw 102 in a closed position to hold the clothing securely, while the lever 106 may allow the provider to open the jaw 102 when attaching or removing the wearable clip for fluid containers 100. This arrangement may enable the provider to administer fluids to a patient through gravity-assisted flow while retaining full use of both hands to render aid, transport the patient, or perform other tasks associated with patient care.

Accordingly, it will be appreciated from the foregoing that the wearable clip for fluid containers 100 disclosed herein solves the need for an apparatus that allows a provider to maintain an IV bag elevated in relation to a patient, that does not require the provider to use a hand to hold the IV bag, and that allows the provider to freely move, among other benefits. This allows for fewer providers to provide care to a patient, reduces the risk of injury to a provider, and allows enhanced care to a patient, which overcomes the limitations of the prior art.

While IV bags have been used as an example throughout, it will be appreciated that the present disclosure is not so limited, and that other fluid containers may be used, including simple hydration containers or bags with a fluid tube or straw attached, among others.

It will be appreciated that systems and methods according to certain embodiments of the present disclosure may include, incorporate, or otherwise comprise properties or features (e.g., components, members, elements, parts, and/or portions) described in other embodiments. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment unless so stated. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Exemplary embodiments are described above. No element, act, or instruction used in this description should be construed as important, necessary, critical, or essential unless explicitly described as such. Although only a few of the exemplary embodiments have been described in detail herein, those skilled in the art will readily appreciate that many modifications are possible in these exemplary embodiments without materially departing from the novel teachings and advantages herein. Accordingly, all such modifications are intended to be included within the scope of this invention.

1. A wearable clip for fluid containers, comprising:
a jaw comprising a first member and a second member;
a plurality of teeth disposed in the jaw, wherein a first row of teeth is disposed on the first member and a second row of teeth is disposed on the second member, the first row of teeth and the second row of teeth configured to mate with one another when the jaw is in a closed position;
a hinge interposed between the first member and the second member;
a lever configured to actuate the jaw about the hinge;
a spring configured to bias the jaw in the closed position; and
a bag support coupled to the second member and configured to receive a fluid container.

2. The wearable clip for fluid containers of claim 1, wherein the bag support comprises a carabiner.

3. The wearable clip for fluid containers of claim 2, wherein the carabiner is coupled to a second arm of the second member.

4. The wearable clip for fluid containers of claim 1, wherein the first member comprises a first arm that extends to form the lever.

5. The wearable clip for fluid containers of claim 4, wherein the second member comprises a second arm, and wherein the hinge is interposed between the first arm and the second arm, the hinge functioning as a fulcrum for the lever.

6. The wearable clip for fluid containers of claim 5, wherein the second arm extends beyond the hinge and is coupled to the bag support.

7. The wearable clip for fluid containers of claim 1, wherein the spring is positioned at the hinge.

8. The wearable clip for fluid containers of claim 1, wherein the jaw is configured to clamp onto an article of clothing worn by a healthcare provider.

9. The wearable clip for fluid containers of claim 8, wherein the article of clothing comprises a jacket, a shirt, or a vest.

10. The wearable clip for fluid containers of claim 1, wherein the fluid container comprises an intravenous solution bag.

11. The wearable clip for fluid containers of claim 1, wherein the bag support extends downward from the second member.

12. A method of using a wearable clip for fluid containers, comprising:
actuating a lever of the wearable clip to open a jaw of the wearable clip, the jaw comprising a plurality of teeth;
placing an article of clothing between the teeth of the jaw;
releasing the lever, wherein a spring forces the jaw closed, thereby clamping the article of clothing within the jaw; and
coupling a fluid container to a bag support of the wearable clip.

13. The method of claim 12, wherein the bag support comprises a neck and a head.

14. The method of claim 13, wherein the neck and head are positioned on a carabiner, and further comprising placing tubing through the carabiner during patient transport.

15. The method of claim 12, wherein the fluid container comprises an intravenous solution bag.

16. The method of claim 12, wherein the article of clothing is worn by a healthcare provider, and wherein the wearable clip is secured at an elevated position relative to a patient such that gravity facilitates fluid flow from the fluid container to the patient.

17. A wearable clip for fluid containers, comprising:
a clamping mechanism configured to releasably secure the wearable clip to an article of clothing;
a lever configured to actuate the clamping mechanism between an open position and a closed position;
a spring configured to bias the clamping mechanism toward the closed position; and
a bag support extending from the clamping mechanism and configured to support a fluid container at a position below the clamping mechanism.

18. The wearable clip for fluid containers of claim 17, wherein the clamping mechanism comprises a jaw having a plurality of teeth configured to grip the article of clothing when the clamping mechanism is in the closed position.

19. The wearable clip for fluid containers of claim 18, wherein the jaw comprises a first member having a first row of teeth and a second member having a second row of teeth, the first row of teeth and the second row of teeth configured to mate with one another when the jaw is in the closed position.

20. The wearable clip for fluid containers of claim 17, wherein the bag support comprises a carabiner configured to receive tubing of the fluid container.
